(19) United States
(12) Patent Application Publication
(10) Pub. No.: US 2021/0321980 A1
(43) Pub. Date: Oct. 21, 2021

(54) IN-BODY CAVITY ULTRASONIC PROBE

(71) Applicant: Hitachi, Ltd., Tokyo (JP)

(72) Inventor: Nobutaka MIYAMOTO, Tokyo (JP)

(21) Appl. No.: 17/192,036

(22) Filed: Mar. 4, 2021

(30) Foreign Application Priority Data
Apr. 20, 2020 (JP) 2020-074497

(51) Int. Cl. A61B 8/12; A61B 8/00

(52) U.S. Cl. CPC A61B 8/12; A61B 8/4444

(57) ABSTRACT

A shaft of a probe that is an in-body cavity ultrasonic probe has a puncture attachment including a needle and a needle guide member. The needle guide member has a straight tubular insertion member through which the needle is inserted. The needle guide member is attached to the shaft rotatably about a guide shaft extending in the lateral direction of the shaft. Since the needle guide member rotates about the guide shaft, the insertion member rotates together, thereby changing the insertion direction of the needle.

### CROSS REFERENCE TO RELATED APPLICATION

This application claims priority to Japanese Patent Application No. 2020-074497 filed on Apr. 20, 2020, which is incorporated herein by reference in its entirety including the specification, claims, drawings, and abstract.

### TECHNICAL FIELD

This description discloses an in-body cavity ultrasonic probe, particularly an in-body cavity ultrasonic probe provided with a needle to be inserted to a test site.

### BACKGROUND

An ultrasonic probe transmits and receives ultrasonic waves to and from a subject. The ultrasonic probe is connected to an ultrasonic diagnostic apparatus body (hereinafter referred to as “apparatus body”), transmits ultrasonic waves to the subject according to signals from the apparatus body, and transmits to the apparatus body electric signals responsive to reflected waves from the subject. The apparatus body forms and displays ultrasonic images based on the electric signals from the ultrasonic probe.

There are various types of ultrasonic probes. For example, there is an in-body cavity ultrasonic probe, a part of which is inserted into the body cavity of a subject and irradiates the test site with ultrasonic waves from the inside of the body cavity. Examples of the in-body cavity ultrasonic probe include transrectal probes, transvaginal probes, and transesophageal probes.

Some in-body cavity ultrasonic probes have a needle to be inserted at a test site for the purpose of collecting tissue at the test site, injecting a drug into the test site, or treating the test site.

Conventionally, a technique for an in-body cavity ultrasonic probe with a needle has been proposed in which the insertion direction of the needle is made adjustable. For instance, JP 2000-139914 A discloses an in-body cavity ultrasonic probe with a needle, having a riser at the lower part of the needle outlet, so that the riser rises and the needle is pushed up and bent by the riser for adjustment of the insertion direction of the needle.

Although in-body cavity ultrasonic probes in which the insertion direction of the needle is adjustable as described above have been conventionally proposed, the insertion direction in conventional in-body cavity ultrasonic probes is adjusted by bending the needle.

It is an advantage of the in-body cavity ultrasonic probe disclosed in this description to be an in-body cavity ultrasonic probe with a needle whose insertion direction can be adjusted without bending the needle.

### SUMMARY

An in-body cavity ultrasonic probe according to this disclosure includes: an ultrasonic oscillator that is to be inserted into a body cavity of a subject and transmits ultrasonic waves to a test site; a shaft that has a slim shape and is to be inserted into the body cavity; a needle to be inserted to the test site; and a needle guide member that includes a straight tubular insertion member through which the needle is inserted, has a slim shape, and is to be mounted to the shaft, wherein the needle guide member rotates about a guide shaft extending in the lateral direction of the shaft, thereby changing the insertion direction of the needle.

### Advantageous Effects of Invention

According to the in-body cavity ultrasonic probe disclosed in this description, in the in-body cavity ultrasonic probe with a needle, the insertion direction of the needle can be adjusted without bending the needle.

### BRIEF DESCRIPTION OF DRAWINGS

An embodiment of the present disclosure will be described based on the following figures, wherein:

FIG. 1 is an external perspective view of a probe according to an embodiment;

FIG. 2 is a side view of the probe according to the embodiment;

FIG. 3 is a bottom view of the probe according to the embodiment;

FIG. 4 is a diagram showing how the insertion direction of the needle is changed;

FIG. 5 is a side view of the probe showing a modification of a locking mechanism;

FIG. 6 is a bottom view of the probe showing the modification of the locking mechanism;

FIG. 7 is a diagram showing the relationship between the planes of ultrasonic radiation from two acoustic heads and the insertion direction of the needle;

FIG. 8 is a diagram of presentation of a puncture guide superimposed on an ultrasonic image A; and

FIG. 9 is a diagram of presentation of a puncture guide superimposed on an ultrasonic image B.

### DESCRIPTION OF EMBODIMENTS

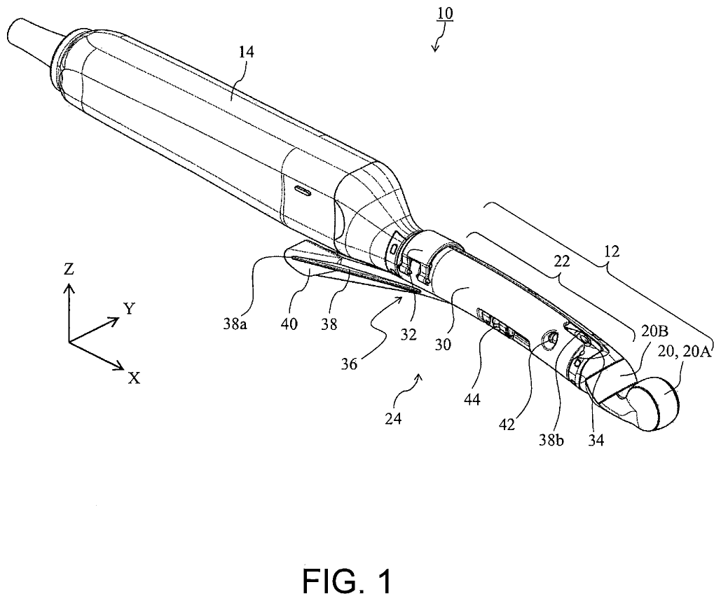

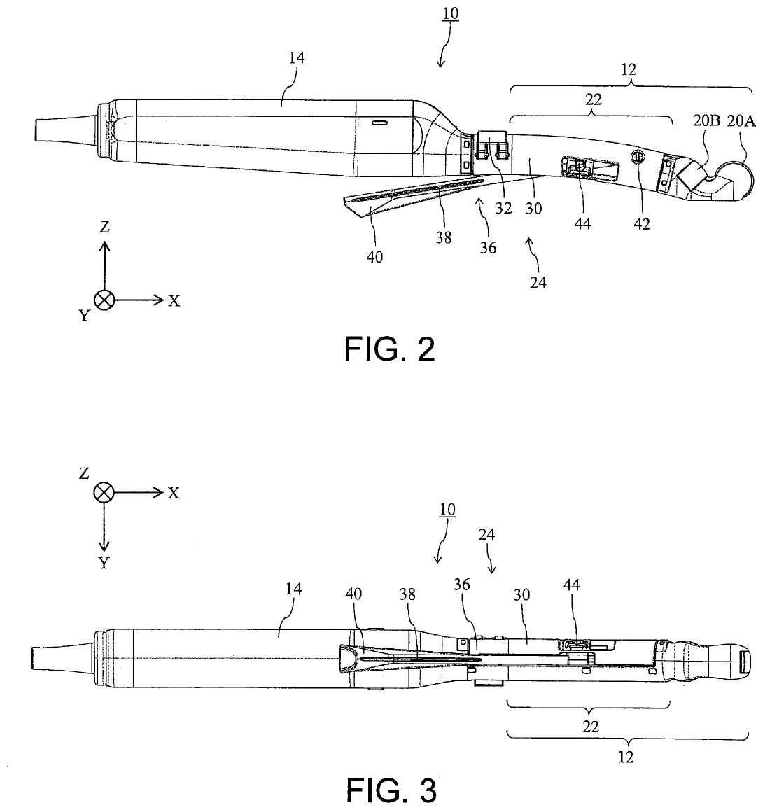

FIG. 1 is an external perspective view of a probe 10 according to an embodiment, FIG. 2 is a side view of the probe 10, and FIG. 3 is a bottom view of the probe 10. The probe 10 is an in-body cavity ultrasonic probe, a part of which is inserted into the body cavity of a subject and sends and receives ultrasonic waves to and from a test site from the inside of the body cavity. No particular limitation is imposed on the in-body cavity ultrasonic probe according to this disclosure, but the probe 10 in this embodiment is a transrectal probe inserted into the rectum of a subject. In particular, the test site for the probe 10 is the prostate of the subject. The probe 10 generally has a slim shape. In FIGS. 1, 2, and 3, the X axis represents the longitudinal direction (stretching direction) of the probe 10, the Y axis represents the lateral direction of the probe 10, and the Z axis represents the height direction. In this description, “upper” refers to the positive side defined with respect to the Z-axis direction, and “lower” refers to the negative side defined with respect to the Z-axis direction.

The probe 10 is connected to an apparatus body (not shown in the drawing) through a probe cable (not shown in the drawing). Note that the probe 10 may be wirelessly connected to the apparatus body so that it can communicate with the apparatus body. The probe 10 transmits ultrasonic waves to the subject according to signals from the apparatus body, and transmits to the apparatus body electric signals responsive to the reflected waves from the subject. The apparatus body forms ultrasonic images based on the electric signals from the ultrasonic probe and displays the formed ultrasonic images on a display provided to the apparatus body.

The probe 10 includes an insertion unit 12 inserted into the body cavity of the subject, and a grip unit 14 gripped by an operator such as a doctor. In this description, the insertion unit 12 side of the probe 10 is referred to as the “distal” side, and the grip unit 14 side is referred to as the “proximal” side. Both the insertion unit 12 and the grip unit 14 have a slim shape, but the insertion unit 12 is made thin for ease of insertion into the body cavity, and the grip unit 14 is made thicker than the insertion unit 12 for ease of gripping by the operator. A probe cable extends from the proximal end of the grip unit 14 toward the apparatus body.

An acoustic head 20 is provided at the distal end of the insertion unit 12. The acoustic head 20 has an ultrasonic oscillator that transmits ultrasonic waves to the test site. In this embodiment, the acoustic head 20 has an oscillator array of aligned ultrasonic oscillators. The oscillator array converts, according to a signal from the apparatus body, the signal into ultrasonic waves, transmits the signal to the outside of the acoustic head 20; that is, the test site, receives the reflected waves from the test site, and converts the reflected waves to an electrical signal.

In this embodiment, the probe 10 has two acoustic heads 20A and 20B. To be specific, the two acoustic heads 20A and 20B are aligned in the direction of the X axis, with the acoustic head 20A provided on the distal side, and the acoustic head 20B provided on the proximal side. As will be described in detail later, the plane of ultrasonic radiation from the acoustic head 20A and the plane of ultrasonic irradiation from the acoustic head 20B are orthogonal to each other. To be specific, the plane of ultrasonic irradiation from the acoustic head 20A is parallel to the XZ plane, and the plane of ultrasonic irradiation from the acoustic head 20B is orthogonal to the XZ plane. Since the probe 10 has the two acoustic heads 20A and 20B, ultrasonic images of two surfaces related to the test site (prostate in this embodiment) can be captured. This allows the operator to easily grasp the test site in three dimensions.

The section from the proximal end of the insertion unit 12 to the proximal end of the acoustic head 20B is a slim, generally cylindrical shaft 22.

The shaft 22 has a notch that is largely notched in the X axis. A puncture attachment 24 is attached to (fitted in) the notch. The puncture attachment 24 is a disposable member (single-use member) and is specifically detachably attached to the shaft 22.

The puncture attachment 24 includes a cover 30. In the state where the puncture attachment 24 is attached to the shaft 22 (hereinafter referred to as “attached state”), the shaft 22 and the puncture attachment 24 form a generally cylindrical shape together. To be specific, the cover 30 has a curved shape and, in the attached state, the outer surface of the shaft 22 and the outer surface of the cover 30 are substantially flush with each other, so that the overall cover 30 has a generally cylindrical shape. In particular, in the attached state, no protrusion is formed sideward from the shaft 22. This facilitates insertion of the shaft 22 into the body cavity of the subject and suppresses the invasion of the subject.

As described below, the puncture attachment 24 includes a plurality of members that are generally situated within the cover 30 in the attached state. In the attached state, the attached state is maintained by an attachment lock 32 provided on the proximal side from the shaft 22.

The puncture attachment 24 includes a needle 34 and a needle guide member 36. The needle 34 is inserted into the test site by operator's operation for the purpose of collecting tissue of the test site, injecting a drug into the test site, or treating the test site. The needle 34 is made of a metal such as stainless steel.

The needle guide member 36 generally has a slim shape and is provided so as to extend in the X axis direction with a slight inclination so that its proximal end is lower than its distal end. The distal end of the needle guide member 36 is fixed within the cover 30 of the shaft 22, and the distal portion of the needle guide member 36 is situated within the cover 30. A notch for passing the needle guide member 36 is provided in a lower portion of the cover 30, and the proximal portion of the needle guide member 36 extends out downward from around the proximal end of the shaft 22 toward the distal side. The proximal end of the needle guide member 36 reaches the lower part of the grip unit 14. The proximal portion of the needle guide member 36 is not inserted into the body cavity of the subject.

The needle guide member 36 has a straight tubular shape and includes an insertion member 38 through which the needle 34 is inserted, and a resin member 40 that supports the insertion member 38. The insertion member 38 and the resin member 40 are bonded to each other. The insertion member 38 defines the puncture route of the needle 34, and is made of a highly rigid member, for example, a metal such as stainless steel. The needle 34 is inserted into the insertion member 38 from the proximal end 38a of the insertion member 38, and goes out toward the distal side from the distal end 38b of the insertion member 38. Since the insertion member 38 has a straight tubular shape and is a highly rigid member, bending of the needle 34 passing therethrough is suppressed. The resin member 40 is a member having lower rigidity than the insertion member 38 and composed of, for example, a resin such as plastic. As will be described later, the needle guide member 36 is operated by the operator, and the resin member 40 is a portion gripped by the operator during operation.

The distal end of the needle guide member 36 is fixed with a guide shaft 42 extending in the lateral direction of the shaft 22 (that is, the direction of the Y axis). As a result, the needle guide member 36 is attached to the shaft 22 rotatably about the guide shaft 42 in the XZ plane. As described above, in the needle guide member 36, since the insertion member 38 and the resin member 40 are bonded to each other, rotation of the needle guide member 36 causes the insertion member 38 and the resin member 40 to rotate together about the guide shaft 42. Rotation of the insertion member 38 changes the inclination of the insertion member 38 in the XZ plane. Accordingly, the insertion direction of the needle 34 is changed.

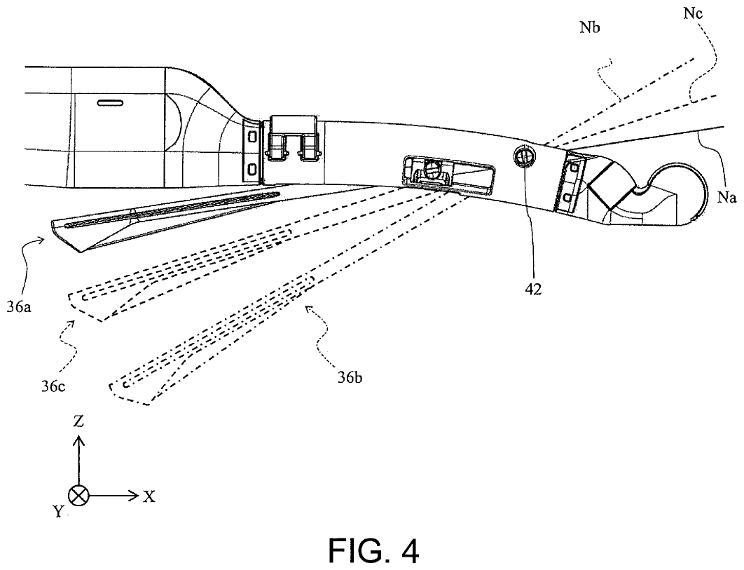

FIG. 4 shows how rotation of the needle guide member 36 changes the insertion direction of the needle 34. For instance, when the inclination of the needle guide member 36 is relatively small and the inclination of the needle guide member 36 is as indicated by reference numeral 36a in FIG. 4, the insertion direction of the needle 34 with respect to the X axis is a relatively small angle which is indicated by reference numeral Na. When the inclination of the needle guide member 36 is relatively large and the inclination of the needle guide member 36 is as indicated by reference numeral 36b in FIG. 4, the insertion direction of the needle 34 with respect to the X axis is a relatively large angle which is indicated by reference numeral Nb. When the inclination of the needle guide member 36 is the inclination indicated by reference numeral 36c between reference numerals 36a and 36b, the insertion direction of the needle 34 is as indicated by reference numeral Nc between reference numerals Na and Nb.

The needle guide member 36 is rotated by the operator. In other words, the operator can change the insertion direction of the needle 34 by rotating the needle guide member 36. In particular, according to this embodiment, the straight tubular insertion member 38 rotates with the rotation of the needle guide member 36. This means that the entire puncture route of the needle 34 is rotated, so that the operator can puncture the test site without bending the needle 34.

The puncture attachment 24 may have a locking mechanism that restricts the rotation of the needle guide member 36 and affirms the insertion direction of the needle 34. In this embodiment, the locking mechanism can lock the needle guide member 36 in any of a plurality of predetermined locking positions (a plurality of inclinations of the needle guide member 36). In other words, the locking mechanism can restrict the rotation of the needle guide member 36 so that the insertion direction of the needle 34 becomes any one of the plurality of predetermined insertion directions.

In the examples shown in FIGS. 1 to 3, a guide holding member 44 that restricts the rotation of the needle guide member 36 is provided as a locking mechanism. Although various methods can be adopted for restricting the rotation of the needle guide member 36 using the guide holding member 44, in this embodiment, the guide holding member 44 laterally holds the resin member 40 of the needle guide member 36, thereby restricting the rotation of the needle guide member 36. The guide holding member 44 is movable along the direction in which the shaft 22 extends and can be locked in any of the plurality of predetermined positions. The needle guide member 36 can be locked in one of the plurality of predetermined locking positions when the guide holding member 44 holds the needle guide member 36 in the position.

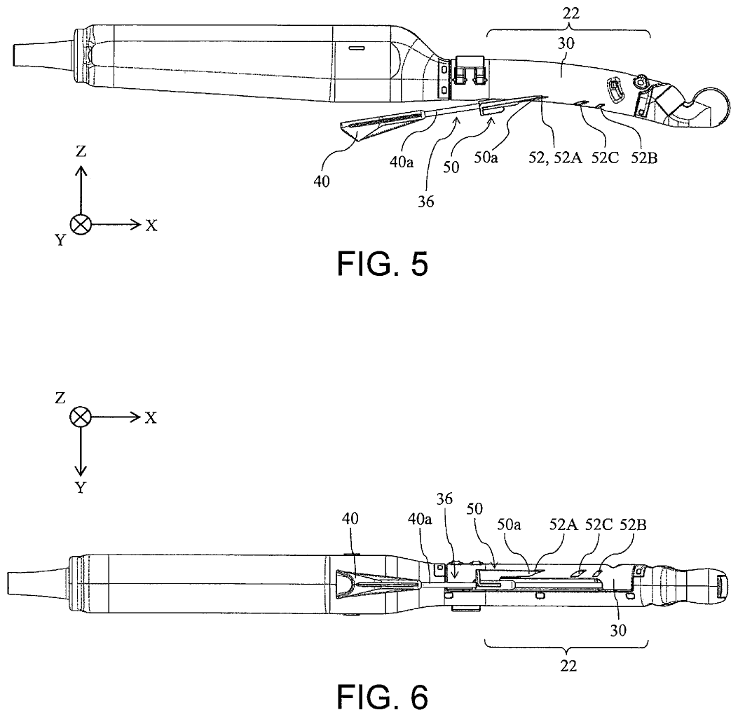

FIG. 5 is a side view of a modification of the locking mechanism, and FIG. 6 is a bottom view of the modification. In the modification, the resin member 40 of the needle guide member 36 has a tubular portion 40a, and a lock pin 50 is provided on the tubular portion 40a. The lock pin 50 is slidable along the tubular portion 40a. A plurality of lock holes 52 extending in the direction in which the shaft 22 extends are provided in the bottom surface (lower surface) of the cover 30.

Inserting the distal end 50a of the lock pin 50 into any of the lock holes 52 restricts the rotation of the needle guide member 36. The needle guide member 36 can be locked in any of the plurality of predetermined locking positions. For example, the distal end 50a of the lock pin 50 inserted in, of the plurality of lock holes 52, the lock hole 52A located on the proximal side is locked at a first insertion direction that is an insertion direction of the needle 34 having a relatively small angle with respect to the X axis. The distal end 50a of the lock pin 50 inserted in, of the plurality of lock holes 52, the lock hole 52B located on the distal side is locked at a second insertion direction that is an insertion direction of the needle 34 having a relatively large angle with respect to the X axis. The distal end 50a of the lock pin 50 inserted in the lock hole 52C situated between the lock hole 52A and the lock hole 52B is locked at a third insertion direction that is an insertion direction of the needle 34 having an angle with respect to the X axis and is situated between the first insertion direction and the second insertion direction. As described above, in the modification, the lock pin 50 and the lock hole 52 constitute a locking mechanism. With the plurality of lock holes 52, when the lock pin 50 is in the lock hole 52, the lock pin 50 may be urged toward the distal side to prevent the lock pin 50 from coming off the lock hole 52 despite the operator's intention.

As described above, the locking mechanism can restrict the rotation of the needle guide member 36 so that the insertion direction of the needle 34 becomes any one of the plurality of predetermined insertion directions. Here, in the case where the insertion direction of the needle 34 in the locking mechanism can be fixed to any one of three or more predetermined insertion directions, the angles between the predetermined insertion directions may be either the same or different.

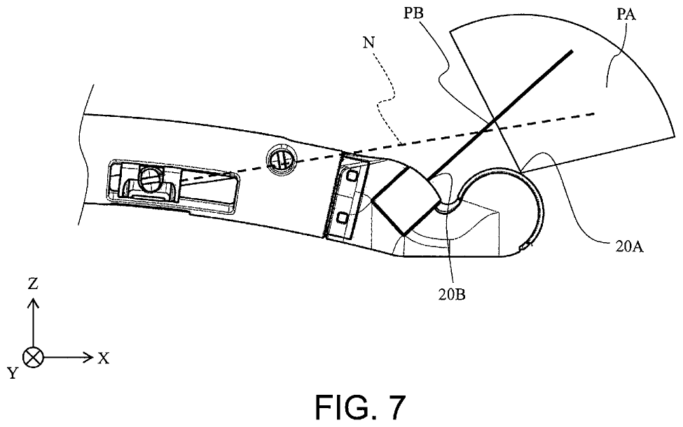

FIG. 7 is a diagram showing the relationship between the plane PA of ultrasonic radiation from the acoustic head 20A and the plane PB of ultrasonic radiation from the acoustic head 20B, and the predetermined insertion direction N of the needle 34. As described above, the plane PA of ultrasonic radiation from the acoustic head 20A is parallel to the XZ plane, the plane PB of ultrasonic radiation from the acoustic head 20B is orthogonal to the XZ plane, and the insertion direction N of the needle 34 is changeable, although the insertion directions N are all parallel to the XZ plane. Hence, in the ultrasonic image A formed by the ultrasonic waves transmitted and received by the acoustic head 20A, how the needle 34 is inserted in the insertion direction N is expressed as if it were viewed from the direction of the Y axis. In the ultrasonic image B formed by the ultrasonic waves transmitted and received by the acoustic head 20B, it is expressed as if it were viewed from the insertion direction N.

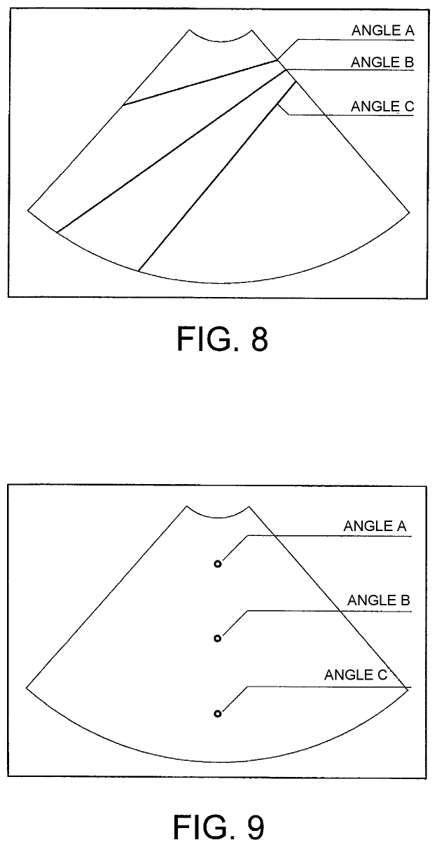

For the apparatus body, puncture guides showing a plurality of predetermined insertion directions defined by the locking mechanism may be superposed on the ultrasonic image A and the ultrasonic image B, and displayed. FIG. 8 shows displayed puncture guides superimposed on the ultrasonic image A. In the ultrasonic image A, the puncture guides representing a plurality of predetermined insertion directions are each shown by a line such as a straight line or dotted line. FIG. 9 shows displayed puncture guides superimposed on the ultrasonic image B. In the ultrasonic image B, the puncture guides representing a plurality of predetermined insertion directions are each shown by a dot or the like.

Although the embodiment of the in-body cavity ultrasonic probe according to this disclosure has been described above, the in-body cavity ultrasonic probe according to this disclosure is not limited to the aforementioned embodiment and various modifications can be made without departing from the scope of this disclosure.

1. An in-body cavity ultrasonic probe comprising:
an ultrasonic oscillator that is to be inserted into a body cavity of a subject and transmits ultrasonic waves to a test site;
a shaft that has a slim shape and is to be inserted into the body cavity;
a needle to be inserted to the test site; and
a needle guide member that includes a straight tubular insertion member through which the needle is inserted, has a slim shape, and is to be mounted to the shaft, wherein
the needle guide member rotates about a guide shaft extending in the lateral direction of the shaft, thereby changing the insertion direction of the needle.

2. The in-body cavity ultrasonic probe according to claim 1, further comprising a locking mechanism that restricts rotation of the needle guide member to lock the insertion direction of the needle.

3. The in-body cavity ultrasonic probe according to claim 2, wherein the locking mechanism can lock the needle guide member in any of a plurality of predetermined locking positions.

4. The in-body cavity ultrasonic probe according to claim 1, wherein a puncture attachment including the needle and the needle guide member is detachably attached to the shaft.
